(19) United States
(12) Patent Application Publication
(10) Pub. No.: US 2017/0051002 A1
(43) Pub. Date: Feb. 23, 2017

(54) Rebaudioside A Crystal And Its Preparation Method And Use

(71) Applicant: Zhucheng Haotian Pharm Co., Ltd., Shandong (CN)

(72) Inventors: Liping Zhu, Zhucheng City (CN); Xuefeng Mei, Shanghai (CN)

(73) Assignee: Zhucheng Haotian Pharm Co., Ltd., Zhucheng City (CN)

(21) Appl. No.: 15/115,542

(22) PCT Filed: Jun. 27, 2014
(86) PCT No.: PCT/CN14/80909

§ 371 Date: Jul. 29, 2016

(30) Foreign Application Priority Data
Jan. 30, 2014 (CN) 201410044560.7

(51) Int. Cl. C07H 15/256; A23L 27/30; C07H 1/06

(52) U.S. Cl. CPC C07H 15/256; C07H 1/06; A23L 27/33; C07B 2200/13; A23V 2002/00

(57) ABSTRACT

Disclosed is a rebaudioside A crystal form 7, of which the structure is represented by the Formula I. The X-ray powder diffraction (XRPD) pattern of the crystal form 7 has the following characteristic peaks at the angles of 2θ±0.1 degrees: 4.80, 5.48, 8.42, 9.27, 11.06, 11.27, 11.86, 12.62, 13.59, 14.20, 15.07, 15.44, 17.05, 17.72, 18.13, 18.62, 19.36, 21.26, 21.95, 22.75, 23.59, 24.14, 24.73, 25.01, 25.54, 25.98, 26.56.

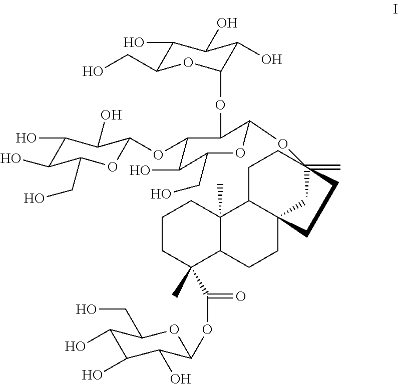

### FIELD OF INVENTION

The present invention relates to the chemical pharmaceutical field, and especially relates to a novel form of rebaudioside A and its preparation method and use.

### BACKGROUND OF INVENTION

Polymorphism is defined as the ability of a compound to exist as more than one crystalline form, each of which has the same chemical structure but different arrangements of the molecules in the crystal lattice. Since different inter- and intramolecular interactions such as van der Waals interactions and hydrogen bonds will be present in different crystal structure, different polymorphs will have different physico-chemical property. In the pharmaceutical research field, the term “polymorph” was used more broadly, including both, solvates and hydrates. Polymorphism is very common in drug research and development, which are mostly connection with the intrinsic property of small organic molecular compounds. In theory, small molecule drugs may have infinite crystal packing patterns, i.e. polymorphic forms. However, the reality studies indicate that the number of polymorphic forms discovered in drugs is directly proportional to the time and resources involved in the R&D process. For example, Lipitor, as a drug having the highest sales in the world till now, has up to 35 crystal forms applied for patents. Polymorphism is not only controlled by intrinsic factors, such as the spatial structure of a molecule itself and performances of functional groups, and intra- and intermolecular interactions, but also is affected by various factors, such as the process design of pharmaceutical synthesis, crystallization and purification conditions, selection of excipients for formulations, formulation technologies, granulation methods, storage conditions, packing materials and the like. Different crystal forms have different processability and physico-chemical properties, such as colors, melting points, solubility, dissolution performances, chemical stability, reactivity, mechanical stability and the like, which may directly affect the safety and effective properties of a drug. Hence, the research and control of crystal forms are research contents of great importance during the drug development.

Normally, the research of polymorphism includes two stages, i.e. polymorph discovery and selection. In the stage of polymorph discovery, various methods are available to crystallize different polymorphs of a compound, such as cooling from the melt, solvent evaporation crystallization, rapid cooling crystallization, suspension crystallization and other crystallization methods. The drug crystallization can be affected by changing crystallization conditions, for example, external factors such as solvent, temperature, mixing speed and a ratio of suspension solvents. The high-throughput platforms was used as well as hundreds of crystallization tests were prepared, utilizing micro-sample preparative techniques and methods for analysis and testing. New crystal forms are prepared and discovered. In the stage of choosing a form for further development crystal characterization is performed on the crystal forms by various solid characterization means, such as X-ray diffraction, solid-state nuclear magnetic resonance, Raman spectroscopy, infrared spectroscopy, etc. In addition, research on the physico-chemical properties of crystal forms is carried out by DSC, TGA, DVS, HPLC and the like; studies are carried out by comparing different crystal forms in terms of hygroscopicity, chemical stability, stability of physical state, processibility and the like. In the end, the most preferred solid form will be developed.

Rebaudioside A is belongs to the Kaurene diterpenoid glycoside, which is a novel natural sweetener refined and extracted from leaves of the Stevia rebaudinan (Bertoni) plant (“Stevia”). Stevia is a composite herbaceous plant and is native to Brazil and Paraguay. The data from the international sweetener industry show that rebaudioside A has found wide application in the production of foods, beverages and spices in Asian, North American, South American and European Community countries. China is the most major rebaudioside A production country in the world.

Rebaudioside A features a high sweetness and a low calorie; its sweetness is 200 to 300 times of that of cane sugar, while its calorie value is only 1/300 of that of cane sugar. Large number of scientific experiments proved that rebaudioside A is a highly ideal sweetener capable of replacing cane sugar without any toxicity and side effect. In addition, rebaudioside A can be widely used in the industries of foods, beverages, spices, brewing and medicine.

The stability and metabolic pathway of rebaudioside A e have been studied extensively; the safety of rebaudioside A with a high purity has been heavily studied. Since the mid-1990s, crude stevioside extracts have been used as food additives. In 2008, pure rebaudioside A has been approved by the United States Food and Drug Administration as the “GRAS (Generally Recognized as Safe)” grade for the first time.

Four polymorphs, Form 1, 2, 3A, 3B and amorphous of rebaudioside A as well as the method for preparing the same are reported in patent US 20070292582 A1. Form 1, Form 2 and high solubility Form 3 as well as the method for preparing the same are reported in patent WO 2010118218 A1, wherein the Form 2 is the same as the Form 1 reported in patent US 20070292582_A1, and the crystal form after the Form 1 is dried is the same as the Form 3A and 3B in US 20070292582_A1. In addition, in an article entitled “Single Crystal Growth and Structure Determination of the Natural “High Potency” Sweetener Rebaudioside A” in Crystal Growth & Design, the tetrahydrated monomethanolated form of Form III is reported, but this form is unstable. The Form I in this article corresponds to the Form 1 in patent US 20070292582_A1, the Form II corresponds to the Form 3A and 3B in US 20070292582_A1, and the Form IV corresponds to the Form 2 in US 20070292582_A1.

There is an urgent need in the field to provide a novel crystal form that has high crystallinity, solubility and stability.

### SUMMARY OF INVENTION

The present invention is directed towards providing a novel rebaudioside A crystal form.

It is another object of the present invention to provide a method for preparing said novel rebaudioside A crystal form.

It is a further object of the present invention to provide the use of said novel rebaudioside A crystal form.

In a first aspect of the present invention, there is provided a rebaudioside A crystal form 7, of which the structure is represented by the Formula I; the X-ray powder diffraction (XRPD) pattern of the crystal form 7 has characteristic peaks at the following angles of 2θ±0.1 degrees: 4.80, 5.48, 8.42, 9.27, 11.06, 11.27, 11.86, 12.62, 13.59, 14.20, 15.07, 15.44, 17.05, 17.72, 18.13, 18.62, 19.36, 21.26, 21.95, 22.75, 23.59, 24.14, 24.73, 25.01, 25.54, 25.98, 26.56;

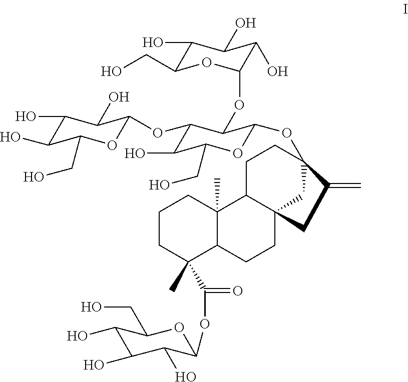

In another preferred embodiment, crystal form 7 has the X-ray powder diffraction (XRPD) pattern as shown in FIG. 1.

In another preferred embodiment, crystal form 7 has no characteristic endothermic peak at 50-250° C. by differential scanning calorimetric analysis.

In another preferred embodiment, the crystal of Form 7 belongs to monoclinic system, wherein the space group is C 1 2 1, the unit cells are as follows: a=34.1571(8) Å, b=8.1098(2) Å, c=19.6378(4) Å, α=γ=90, β=109.6250(1)°, and the volume per formula unit of Form 7 is 5123.8(2) Å³.

In a second aspect of the present invention, there is provided a method for preparing the rebaudioside A crystal form 7 provided by the present invention as described above, the method comprising the steps of:

(1) mixing rebaudioside A with a solvent at 40-90° C. to give a saturated solution;

(2) filtering the saturated solution and taking the clear filtrate;

(3) subjecting the clear filtrate to crystallization of a crystal of rebaudioside A form 7 at minus 20-20° C.

In another preferred embodiment, the filtration in step (2) is carried out at the same temperature as in step (1).

In another preferred embodiment, the clear filtrate is allowed to stand for 1-30 days at minus 20-20° C. in step (3) for crystallization of a crystal of rebaudioside A Form 7.

In another preferred embodiment, the crystal crystallized in step (3) is dried by baking.

In another preferred embodiment, said solvent in step (1) is selected from one or more of water, methanol, ethanol and tetrahydrofuran.

In a third aspect of the present invention, there is provided the use of the rebaudioside A Form 7 provided by the present invention as described above in the manufacture of foods and medicaments.

Accordingly, the present invention provides a novel crystal form having better performance, that has high crystallinity, solubility and stability.

### BRIEF DESCRIPTION OF THE DRAWINGS

FIG. 1 is a powder X-ray diffraction (XRPD) pattern of rebaudioside A polymorph Form 7 obtained from an embodiment.

FIG. 2 is a thermal gravimetric analysis (TG) plot of rebaudioside A Form 7 obtained from the embodiment.

FIG. 3 is a differential scanning calorimetric analysis (DSC) curve of the rebaudioside A Form 7 obtained from the embodiment.

FIG. 4 is a dynamic vapor adsorption (DVS) isotherms of the rebaudioside A Form 7 obtained from the embodiment.

FIG. 5 is an infrared (IR) spectroscopy of rebaudioside A Form 7 obtained from the embodiment.

FIG. 6 is a Raman spectroscopy of rebaudioside A Form 7 obtained from the embodiment.

FIG. 7 is a single crystal structure of rebaudioside A Form 7 obtained from the embodiment.

### DETAILED DESCRIPTION OF INVENTION

Attempts have been made by the inventors to find a novel rebaudioside A polymorph form, i.e. Form 7, and a simple preparation method of obtaining this form is also discovered. On this basis, the present invention was done.

As used herein, the phrases “compound of Formula I”, “compound of Formula 1” and “Rebaudioside A” may be used interchangeably and are the compound having the structure shown below:

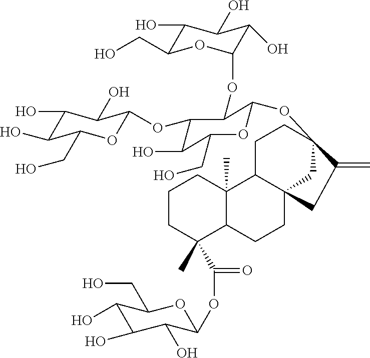 [SMILES: C=C1C[C@@]23CCC4[C@@](C)(CCC[C@@]4(C)C(=O)OC4OC(CO)C(O)C(O)C4O)C2CC[C@]1(OC1OC(CO)C(O)C(OC2OC(CO)C(O)C(O)C2O)C1OC1OC(CO)C(O)C(O)C1O)C3]

The terms “compound”, “composition”, “agent”, “medicine” and “medicament” may be used interchangeably herein and all refer to a compound or composition capable of inducing desired pharmaceutical and/or physiological responses by local and/or systemic effects when administered to an individual (human or animal).

Identification and properties of rebaudioside A Form 7 After obtaining a novel crystal form of rebaudioside A, the inventors have further carried out a research on its properties by various technologies and instruments.

“powder X-ray diffraction”, also referred to as “polycrystalline X-ray diffraction (XRPD)”, is a common method presently used for determining a crystal structure (i.e. a crystal form). A series of diffraction patterns are produced as X-ray transmits a crystal using an X-ray powder diffractometer. Different positions and their intensities in the pattern are determined by the size and shape of the unit cell of the crystalline phase, whereby the specific crystal structure of the crystal is determined.

Methods for determination of powder X-ray diffraction of a crystal are known in the field. For example, an Bruker D8 Advanced X-ray powder diffractometer is used to obtain a pattern using a copper radiation target at a scanning speed of 2° per minute.

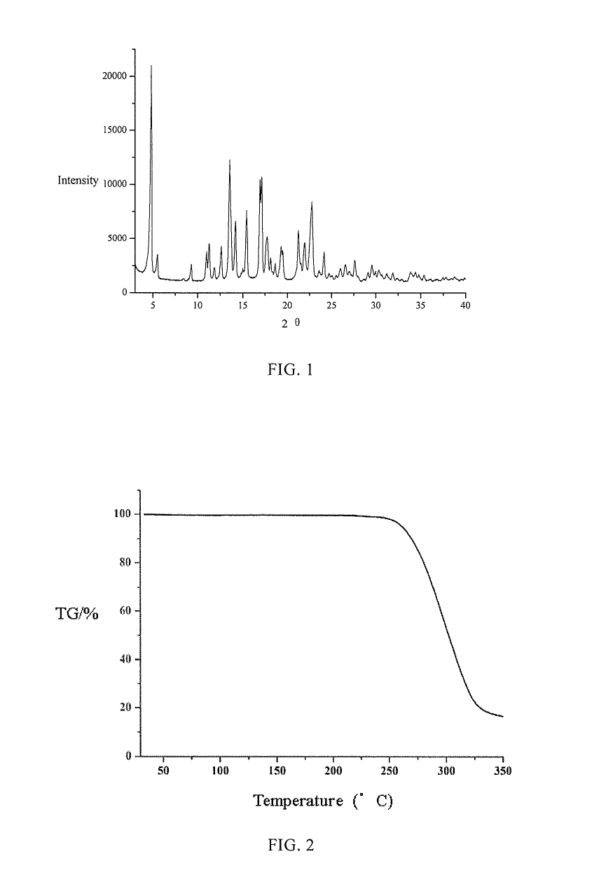

The rebaudioside A Form 7 of the present invention has a specific crystal habit and has specific characteristic peaks in the powder X-ray diffraction (XRPD) pattern. In particular, the powder X-ray diffraction (XRPD) pattern of rebaudioside A Form 7 of the present invention has strong peaks at the following angles of 2θ±0.1 degrees: 4.80, 5.48, 8.42, 9.27, 11.05, 11.27, 11.86, 12.62, 13.59, 14.20, 15.07, 15.44, 17.05, 17.72, 18.13, 18.62, 19.36, 21.26, 21.95, 22.75, 23.59, 24.14, 24.73, 25.01, 25.54, 25.98, 26.56. In a preferred embodiment of the present invention, the rebaudioside A Form 7 has an powder X-ray diffraction (XRPD) pattern substantially identical to FIG. 1.

“Differential scanning calorimetric analysis”, also referred to as “differential scanning calorimetry (DSC)” refers to a technique for measuring a relation of the energy difference and temperature of between a measured substance and a reference substance during a heating process. Peak positions, shapes and peak numbers in DSC thermograms are associated with properties of the substances, and thus this technique can be qualitatively used for identifying substances. This process is commonly used in the field for detecting various parameters of a substance, such as a phase transition temperature, a glass transition temperature and reaction heat.

The DSC measurement method is known in the field. For example, a DSC Q20 differential scanning calorimeter can be used to acquire a DSC scanning spectra of a substance by raising the temperature from 25° C. to 300° C. at a rate of a temperature rise of 10° C. per minute.

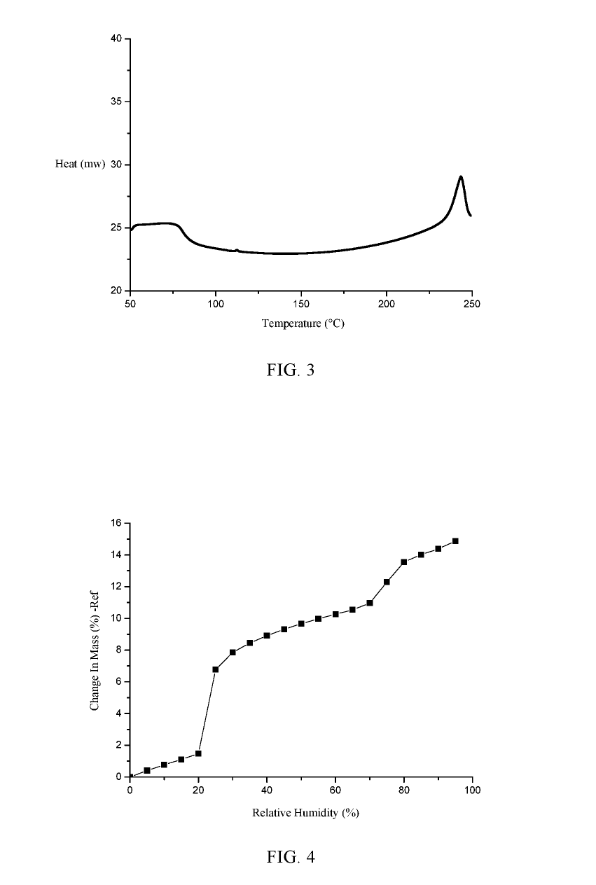

In one embodiment of the present invention, there is no characteristic endothermic peak determined at 50-250° C. for the rebaudioside A Form 7 obtained using the process of the invention by differential scanning calorimetric analysis. It is preferred that the rebaudioside A Form 7 of the invention has a DSC thermogram substantially identical to FIG. 3.

“Thermal gravimetric analysis (TG)” may be used to analyze moisture, volatiles, ash contents, fixed carbon and LOI of an analyte.

The TG measurement method is known in the field. For example, a dynamic vapour adsorption instrument may be used.

In one embodiment of the present invention, the rebaudioside A Form 7 obtained using the process of the invention has a TG pattern substantially identical to FIG. 2, which is measured by TG.

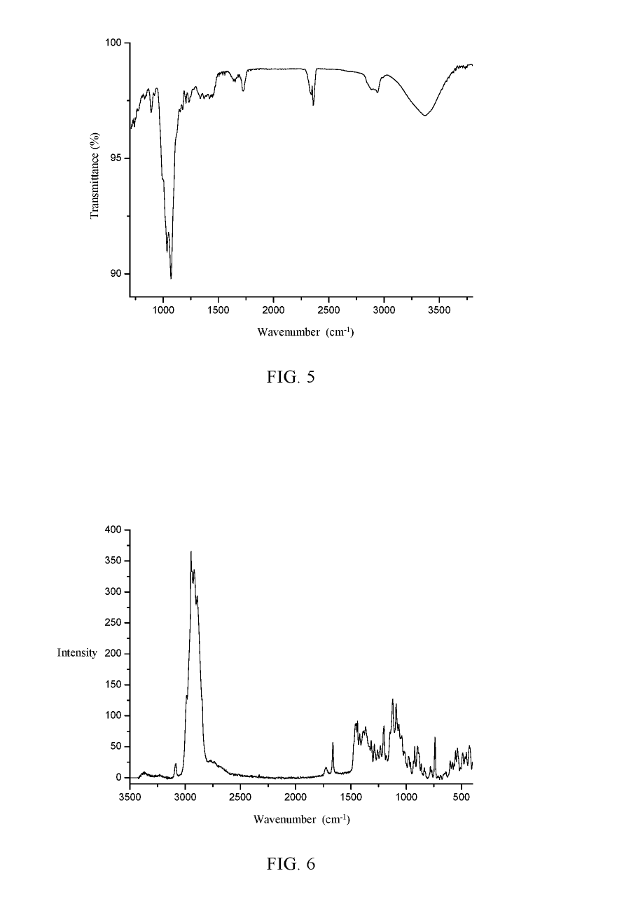

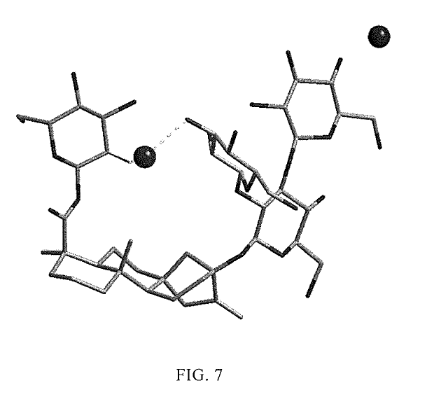

Infrared spectroscopy (IR) may also be utilized for the determination of the crystal structure, and its measurement method is known in the field. For example, PE Spectrum One B may be utilized with KBr:sample=200:1 and scanning within a range from 400 to 4000 cm⁻¹. The rebaudioside A Form 7 of the present invention has an infrared spectroscopy substantially identical to FIG. 5.

Raman characteristic spectroscopy may also be utilized for the determination of the crystal structure, and its measurement method is known in the field. For example, a Thermo Scientific DXR Raman spectrometer may be utilized with a sample being placed on a slide and scanned at a wavelength of 532 nm within a range from 3500 to 50 cm⁻¹. The rebaudioside A Form 7 of the present invention has a characteristic Raman spectroscopy substantially identical to FIG. 6.

“Dynamic vapor adsorption (DVS)” is an instrument for measuring the hygroscopicity of a sample. Its measurement method is known in the field. For example, an instrument from Surface Measurement Systems, Ltd. may be used to collect data of humidity ranging from 5% to 95% at 25 degrees Celsius. The weight deviation under each humidity is no more than ±0.02% within 10 min.

The DVS measurement method is known in thefield. For example, a dynamic vapour adsorption instrument may be used.

The rebaudioside A Form 7 of the present invention has particular stability, which is conducive to storage. The Form 7 is shown to have little hygroscopicity in a conventional storage environment (40%-80% RH) in DVS patterns carried out by the inventors. In a preferred embodiment, the DVS isotherm of the resulting rebaudioside A crystal form 7 is substantially identical to FIG. 4.

The rebaudioside A Form 7 belongs to monoclinic system, wherein the space group is C 1 2 1, the cell parameters are as follows: a=34.1571(8) Å; b=8.1098(2) Å; c=19.6378(4) Å; α=γ=90°; β=109.6250(1)°, and the volume per formula unit is 5123.8(2) Å3.

Preparation Method of Rebaudioside A Form 7

The present invention provides a method for preparing the rebaudioside A Form 7, the method comprising the following steps:

step I: a saturated solution preparation, that is, excess rebaudioside A is dissolved in a solvent at 40-90° C. to give a saturated solution;

step II: the resulting saturated solution from step one is filtered hot and the clear filtrate is taken;

step III: cooling crystallization, that is, the clear filtrate is allowed to stand at a temperature ranging from minus 20° C. to 20° C. for crystallization of rebaudioside A Form 7.

The solvent involved in step I is selected from water, methanol, ethanol, tetrahydrofuran, or a mixture of two or more from these solvents, such as, but not limited to, a mixture of methanol and tetrahydrofuran, a mixture of methanol and ethanol, or a mixture of methanol, ethanol and water. Regarding the mixing ratio of these solvents, the volume ratio of other solvents to methanol may be 0.3-3:1, on the basis of methanol, such as, but not limited to, in a mixed solvent consisting of methanol and tetrahydrofuran, the volume ratio of tetrahydrofuran to methanol is 0.3-3:1, preferably 0.5-2:1; in a mixed solvent consisting of methanol and ethanol, the volume ratio of ethanol to methanol is 0.3-3:1; and in a mixed solvent consisting of methanol, ethanol and water, the volume ratio of ethanol to methanol is 0.3-3:1, preferably 0.6-1:1, and the volume ratio of water to methanol is 0.3-3:1, preferably 0.3-1:1.

The temperature of preparing the saturated solution involved in step I is preferably 50-70° C., more preferably 60° C.

The filtration step involved in step II is preferably carried out at the same temperature as in step I.

The temperature of crystallization involved in step III is preferably minus 10-10° C., more preferably 0-5° C., and most preferably 5° C.

The crystallization in step III is carried out preferably after standing for 3-20 days, more preferably for 7-15 days.

In one embodiment of the present invention, the clear filtrate is allowed to stand at the above indicated temperature in step III, and the resulting solid after centrifugation is dried by baking at ordinary pressure or a reduced pressure at 50° C. to obtain rebaudioside A Form 7.

Use of Rebaudioside A Form 7 and its Composition

The present invention also relates to a composition comprising the novel crystal form of rebaudioside A provided by the present invention, wherein said composition comprises an effective amount of the rebaudioside A Form 7 and a dietary/pharmaceutically acceptable carrier.

As used herein, the term “comprising” or “including” includes “comprise”, “consisting essentially of . . . ” and “consisting of . . . ”. The term “an effective amount” refers to an amount capable of having effects or activity on human and/or animals which is acceptable for human and/or animals.

The term “pharmaceutically acceptable” or “dietary acceptable” constituent is a material suitable for human and/or animals without an excessive adverse side effect (such as toxicity, irritation and allergic reactions), i.e. a material with a reasonable benefit/risk ratio.

Preferably, “pharmaceutically acceptable carrier” is selected from fillers, disintegrants, lubricants, glidants, effervescing agents, flavoring agents, coating materials, excipients or sustained/controlled release agents. In the composition, the pharmaceutically acceptable carrier may comprise liquids, such as water, saline, glycerol and ethanol. In addition, auxiliary substances, such as fillers, disintegrants, lubricants, glidants, effervescing agents, wetting or emulsifying agents, flavoring agents, and pH buffering materials, may also be present in such carrier. Generally, these substances can be formulated into a non-toxic, inert and pharmaceutically acceptable aqueous carrier medium, wherein pH is generally about 5-8, preferably pH is about 6-8.

The above-mentioned features mentioned herein or features mentioned in the embodiments may be combined as desired. All features disclosed in the present description may be used in combination with compositions in any form, and each feature disclosed in the description may be replaced by any alternative feature that can provide identical, equivalent or similar purposes. Thus, the features disclosed herein are only general examples of equivalent or similar features, unless specified otherwise.

The major advantages of the present invention are as follows:

1. The novel crystal form provided by the present invention has high crystallinity, significantly increased solubility, and a certain degree of improvement in chemical and physical stability.

2. The novel crystal form provided by the present invention has excellent chemical and physical stability, and regularly formed crystal morphology, which is conducive to preparation processing of rebaudioside A and its extensive applications in industry.

3. The method for preparing the novel crystal form of rebaudioside A provided by the present invention is simple and is easy for industrial production.

The present invention is further illustrated in connection with the following specific embodiments. It should be understood that these embodiments only serve to illustrate the present invention and are not intended to limit the scope of the present invention. Those test methods without specific conditions specified in the following embodiments are typically carried out according to conventional conditions or according to conditions recommended by the manufacturer. Unless indicated otherwise, all percentages, rates, ratios or parts are by weight.

The units of percent weight in volume in the present invention are well known to those skilled in the field, for example, it refers to the weight of a solute in a solution of 100 milliliters.

Unless otherwise defined, all the technical and scientific terms as used herein have the same meaning familiar to those skilled in the field. In addition, any method and material similar or equivalent to those described herein may be used in the methods of the present invention. The preferred embodiments and materials as described herein are only for the purpose of demonstration.

Experimental Conditions:

XRPD: All XRPD spectrograms of the present invention are detected at room temperature using a Bruker D8 Advance X-ray diffractometer, scanning with angles of 2θ ranging from 3 degrees to 40 degrees, Cu Kα, at a scanning speed of 0.1 degree/step.

It should be noted that in X-ray diffraction patterns of powder samples, a crystal form with a specific diffraction pattern obtained from a crystalline compound is typically characteristic, wherein the relative intensity of the spectral band (particularly at low angles) may vary as preferential orientation effects caused by differences in terms of crystallization conditions, a particle size, a relative amount in the mixture and other test conditions. Hence, the relative intensity of a diffraction peak is not characteristic with respect to a targeted crystal. More attention should be paid to positions of the peaks than their relative intensities in determining whether a crystal is identical to a known crystal form. In addition, holistic thinking is of great importance in determining the identity of crystal forms, because a set of specific data of “d−I/I₁” rather than a single diffraction peak represents a certain physical phase. It should also be noted that in the identification of a mixture, factors, for example a reduced content will result in the loss of a part of diffraction peaks. In this case, even a single spectral band may be characteristic for a given crystal without the need for all spectral bands observed in a high-purity sample.

All DSC spectrograms of the present invention are detected by using a DSC 8500 differential scanning calorimeter from Perkin Elmer, US, with a nitrogen atmosphere, at a heating rate of 10 Celsius degrees/min.

IR: All infrared spectroscopies of the present invention are detected by a Nicolet-Magna FT-IR 750 infrared spectrometer from Nicolet, US, at room temperature, with a detection range of 4000-500 cm⁻¹ wave numbers.

Raman: All Raman spectroscopies of the present invention are detected by a DXR Microscopes Raman Spectrometer from ThermoFisher, US, at room temperature, with a detection range of a Raman shift of 3500-450 cm⁻¹. The wavelength of laser is 532 nm.

DVS: All dynamic vapour adsorption (DVS) experimental data of the present invention are measured by a DVS Intrinsic dynamic vapour sorption instrument from SMS, UK. Measurement conditions: Temperature: 25° C.; Relative humidity range: 5%-95%.

SCXRD: All single crystal X-ray diffraction (XRPD) experimental data in this experiment are measured by a Bruker Smart Apex II X-ray single crystal diffractometer from Bruker Spectrospin Co. Ltd.

Measurement conditions: graphite monochromator, Mo-Ka ray (λ=0.71073 Å); temperature: room temperature; voltage: 50 kilovolts; current: 30 milliamperes. Data reduction and structure analysis of all single crystal structures are completed by SAINT-5.0 and SHELXTL-97 programs, respectively, and absorption correction is completed by an SADABS program. The non-hydrogen atoms coordinates are calculated by the difference function and least square method, and hydrogen atoms are placed at suitable locations by theoretical calculation.

The raw material rebaudioside A in the following examples are commercially available from Zhucheng Haotian Pharm Co., Ltd.

### EXAMPLE 1

At 60° C., excess rebaudioside A was dissolved in methanol-THF (1:1) and filtered, and 1 milliliter of the clear filtrate was taken; the filtrate was allowed to stand for 7 days at 5° C. and dried by baking at ordinary pressure at 50° C. to obtain rebaudioside A Form 7.

### EXAMPLE 2

At 60° C., excess rebaudioside A was dissolved in methanol-THF (2:1) and filtered, and 1 milliliter of the clear filtrate was taken; the filtrate was allowed to stand for 7 days at 5° C. and dried by baking at ordinary pressure at 50° C. to obtain rebaudioside A Form 7.

### EXAMPLE 3

At 60° C., excess rebaudioside A was dissolved in methanol-THF (1:2) and filtered, and 1 milliliter of the clear filtrate was taken; the filtrate was allowed to stand for 15 days at 5° C. and dried by baking at ordinary pressure at 50° C. to obtain rebaudioside A Form 7.

### EXAMPLE 4

At 60° C., excess rebaudioside A was dissolved in methanol-ethanol (1:1) and filtered, and 1 milliliter of the clear filtrate was taken; the filtrate was allowed to stand for 15 days at 5° C. and dried by baking at ordinary pressure at 50° C. to obtain rebaudioside A Form 7.

### EXAMPLE 5

At 60° C., excess rebaudioside A was dissolved in methanol-ethanol (2:1) and filtered, and 1 milliliter of the clear filtrate was taken; the filtrate was allowed to stand for 15 days at 5° C. and dried by baking at ordinary pressure at 50° C. to obtain rebaudioside A Form 7.

### EXAMPLE 6

At 60° C., excess rebaudioside A was dissolved in methanol-ethanol (1:2) and filtered, and 1 milliliter of the clear filtrate was taken; the filtrate was allowed to stand for 15 days at 5° C. and dried by baking at ordinary pressure at 50° C. to obtain rebaudioside A Form 7.

### EXAMPLE 7

At 60° C., excess rebaudioside A was dissolved in methanol-ethanol (1:3) and filtered, and 1 milliliter of the clear filtrate was taken; the filtrate was allowed to stand for 15 days at 5° C. and dried by baking at ordinary pressure at 50° C. to obtain rebaudioside A Form 7.

### EXAMPLE 8

At 60° C., excess rebaudioside A was dissolved in methanol-ethanol (3:1) and filtered, and 1 milliliter of the clear filtrate was taken; the filtrate was allowed to stand for 15 days at 5° C. and dried by baking at ordinary pressure at 50° C. to obtain rebaudioside A Form 7.

### EXAMPLE 9

At 60° C., excess rebaudioside A was dissolved in methanol-ethanol-water (2:2:1) and filtered, and 1 milliliter of the clear filtrate was taken; the filtrate was allowed to stand for 15 days at 5° C. and dried by baking at ordinary pressure at 50° C. to obtain rebaudioside A Form 7.

### EXAMPLE 10

At 60° C., excess rebaudioside A was dissolved in methanol-ethanol-water (3:2:1) and filtered, and 1 milliliter of the clear filtrate was taken; the filtrate was allowed to stand for 15 days at 5° C. and dried by baking at ordinary pressure at 50° C. to obtain rebaudioside A Form 7.

### EXAMPLE 11

At 60° C., excess rebaudioside A was dissolved in methanol-ethanol-water (3:2:3) and filtered, and 1 milliliter of the clear filtrate was taken; the filtrate was allowed to stand for 15 days at 5° C. and dried by baking at ordinary pressure at 50° C. to obtain rebaudioside A Form 7.

### EXAMPLE 12

At 70° C., excess rebaudioside A was dissolved in methanol-THF (1:1) and filtered, and 1 milliliter of the clear filtrate was taken; the filtrate was allowed to stand for 7 days at 5° C. and dried by baking at ordinary pressure at 50° C. to obtain rebaudioside A Form 7.

### EXAMPLE 13

At 75° C., excess rebaudioside A was dissolved in methanol-THF (1:1) and filtered, and 1 milliliter of the clear filtrate was taken; the filtrate was allowed to stand for 7 days at 5° C. and dried by baking at ordinary pressure at 50° C. to obtain rebaudioside A Form 7.

### EXAMPLE 14

At 70° C., excess rebaudioside A was dissolved in methanol-THF (1:1) and filtered, and 1 milliliter of the clear filtrate was taken; the filtrate was allowed to stand for 7 days at minus 20° C. and dried by baking at ordinary pressure at 50° C. to rebaudioside A Form 7.

### EXAMPLE 15

At 70° C., excess rebaudioside A was dissolved in methanol-THF (1:1) and filtered, and 1 milliliter of the clear filtrate was taken; the filtrate was allowed to stand for 7 days at 20° C. and dried by baking at ordinary pressure at 50° C. to obtain rebaudioside A Form 7.

The maximum solubility of the sample obtained according to the above-mentioned examples is about 30 mg/mL, and the form remains unchanged when stored at 25° C. RH 60% for 3 months from the results of HPLC analysis (purity is detected by high performance liquid chromatography, and the liquid chromatographic column and liquid chromatography used are the same as those specified by JECFA in 2010).

Perparation of sample solution: Precisely 50-100 milligrams of rebaudioside A were weighed and placed in a 50 milliliter volumetric flask, and then a solution of water and acetonitrile in a ratio of 7:3 were added for dissolution to a scale of 50 milliliter. Detection step: 5 μl of a sample solution are injected under the following conditions. Chromatographic Column: A Capcell pak C18 MG II chromatographic column from the Shiseido company or a Luna 5μ C18(2) 100A chromatographic column from the Phenomenex company or a chromatographic column of equivalent specifications (length: 250 millimeters; inner diameter: 4.6 millimeters; packing particle size: 5 μm). Mobile Phase: a mixture of acetonitrile and a sodium phosphate buffer (specification: 10 mmol/L, pH value of 2.6) in a ratio of 32:68. Preparation method of sodium phosphate buffer: 2.76 grams of sodium dihydrogen phosphate were dissolved in 2 liters of water, and phosphoric acid was added to adjust the pH value to 2.6, with a flow rate of 1 milliliter per minute. Detector: A 210 nm ultraviolet detection. Chromatographic Column temperature: detection patterns are recorded for about 30 minutes at 40° C. It is shown that the purity changes by less than 0.5% after standing for 3 months as compared to the starting material.

What specified above are only preferred embodiments of the present invention and are not intended to limit the scope of the substantial technical contents of the invention; the substantial technical contents of the present invention are broadly defined in the scope of the appended claims of the present application; any technology entity or method accomplished by others, if it is virtually identical to that defined in the scope of the appended claims of the present application or it is an equivalent alteration, is considered to be included within the scope of the appended claims.

1. A sweetener rebaudioside A crystal form 7 having an structure represented by Formula I, wherein the X-ray powder diffraction (XRPD) pattern of the crystal form 7 has characteristic peaks at the following angles of 2θ±0.1 degrees: 4.80, 5.48, 8.42, 9.27, 11.06, 11.27, 11.86, 12.62, 13.59, 14.20, 15.07, 15.44, 17.05, 17.72, 18.13, 18.62, 19.36, 21.26, 21.95, 22.75, 23.59, 24.14, 24.73, 25.01, 25.54, 25.98, and 26.56;

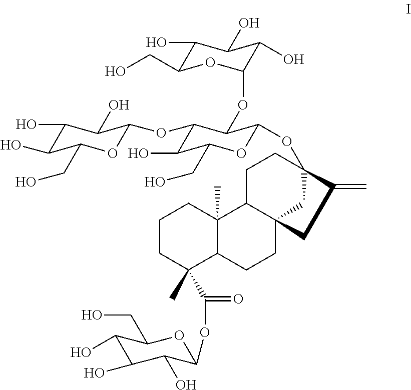

2. (canceled)

3. The steviosiderebaudioside A glycoside crystal form 7 of claim 1, wherein said crystal form 7 has no characteristic endothermic peak at 50-250° C. by differential scanning calorimetric analysis.

4. The rebaudioside A crystal form 7 of claim 1, wherein the crystal form belongs to a monoclinic system, wherein the space group is C 1 2 1, the cell parameters are as follows: a=34.1571(8) Å, b=8.1098(2) Å, c=19.6378(4) Å, α=γ=90°, β=109.6250(1)°, and the unit cell volume is 5123.8(2) Å³.

5. A method for preparing the rebaudioside A crystal form 7 of claim 1, comprising the steps of:
(1) mixing rebaudioside A with a solvent at 40-90° C. to give a saturated solution;
(2) filtering the saturated solution and taking the clear filtrate;
(3) subjecting the clear filtrate to crystallization of the rebaudioside A Form 7 at minus 20-20° C.

6. The method of claim 5, wherein the filtration in step (2) is carried out at the same temperature as in step (1).

7. The method of claim 5, wherein the clear filtrate is allowed to stand for 1-30 days at minus 20-20° C. in step (3) for crystallization of the rebaudioside A Form 7.

8. The method of claim 5, wheren the rebaudioside A Form 7 crystallized in step (3) is dried by baking.

9. The method of claim 5, wheren said solvent in step (1) is selected from one or more of water, methanol, ethanol and tetrahydrofuran.

10. A method of using the rebaudioside A Form 7 of claim 1 comprising, preparing foods or medicaments by incorporating the rebaudioside A Form 7.

11. The rebaudioside A crystal form of claim 3, wherein the crystal form belongs to a monoclinic system, wherein the space group is C 1 2 1, the cell parameters are as follows: a=34.1571(8) Å, b=8.1098(2) Å, c=19.6378(4) Å, α=γ=90°, β=109.6250(1)°, and the unit cell volume is 5123.8(2) Å³.
